(19) United States
(12) Patent Application Publication
(10) Pub. No.: US 2026/0081230 A1
(43) Pub. Date: Mar. 19, 2026

(54) ELECTROLYTIC SOLUTION, AND POWER STORAGE ELEMENT USING SAME

(71) Applicant: Panasonic Intellectual Property Management Co., Ltd., Osaka (JP)

(72) Inventors: NOBUYUKI MATSUZAWA, Tokyo (JP); KEISUKE HAYASHI, Osaka (JP); HIROYUKI MAESHIMA, Hyogo (JP); YOSHIAKI OBANA, Osaka (JP)

(21) Appl. No.: 19/111,561

(22) PCT Filed: Oct. 20, 2023
(86) PCT No.: PCT/JP2023/038067

§ 371 Date: Mar. 13, 2025

(30) Foreign Application Priority Data
Nov. 22, 2022 (JP) 2022-186617

(51) Int. Cl. H01M 10/0569; C07C 13/06; H01M 10/0568

(52) U.S. Cl. CPC H01M 10/0569; C07C 13/06; H01M 10/0568; H01M 2300/0034; H01M 2300/0037

(57) ABSTRACT

Electrolytic solution contains non-aqueous solvent and electrolyte salt dissolved in the non-aqueous solvent. The non-aqueous solvent contains a first compound, and the first compound is at least one type selected from a group including 1,3-diethyl-4-methyl-1-cyclobutene and a fluoro-substituted compound of the 1,3-diethyl-4-methyl-1-cyclobutene. An electricity storage element is configured using this electrolytic solution.

### TECHNICAL FIELD

The present disclosure relates to electrolytic solution and an electricity storage element using the electrolytic solution.

### BACKGROUND ART

An electricity storage element is used in various applications. For example, an electric double layer capacitor and a lithium ion capacitor are used as a small power supply or the like used for backup of a semiconductor memory or the like. Since these capacitors are assumed to be used under severe conditions, characteristics capable of stably operating the capacitors for a long period of time in a wide temperature range from low temperature to high temperature are important for electrolytic solution to be used.

PTL 1 discloses electrolytic solution for an electric double layer capacitor in which tetraethylammonium tetrafluoroborate which is aliphatic quaternary ammonium salt is dissolved as electrolyte salt in propylene carbonate which is organic solvent.

PTL 2 discloses electrolytic solution for a capacitor using quaternary ammonium salt or lithium salt as electrolyte salt and using mixed solvent containing acetonitrile as organic solvent. Acetonitrile is characterized in that its viscosity at room temperature is as very low as 0.34 mPa·s, and therefore has a characteristic of being able to reduce a resistance value at low temperature of an element, in particular.

The propylene carbonate and the acetonitrile are also used as electrolytic solution of a nonaqueous electrolytic secondary battery.

### CITATION LIST

### Patent Literature

PTL 1: Unexamined Japanese Patent Publication No. 2000-114105
PTL 2: International Publication No. WO 2013/146136

### SUMMARY OF THE INVENTION

One aspect of the present disclosure relates to electrolytic solution including a non-aqueous solvent, and an electrolyte salt dissolved in the non-aqueous solvent, the non-aqueous solvent contains a first compound, and the first compound is at least one selected from the group consisting of 1,3-diethyl-4-methyl-1-cyclobutene represented by the following Formula (1) and its fluoro-substituted compound.

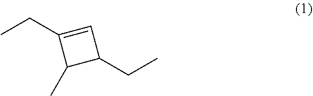

Another aspect of the present disclosure relates to an electricity storage element including the above electrolytic solution.

Yet another aspect of the present disclosure relates to at least one compound selected from the group consisting of 1,3-diethyl-4-methyl-1-cyclobutene and its fluoro-substituted compound.

According to the electrolytic solution of the present disclosure, it is possible to provide an electricity storage element which can exhibit an excellent electrical characteristic with low resistance particularly at low temperature.

### BRIEF DESCRIPTION OF THE DRAWINGS

FIG. 1 is a partially cut-away perspective view schematically illustrating an internal structure of a secondary battery according to an exemplary embodiment of the present disclosure.

### DESCRIPTION OF EMBODIMENT

Prior to description of an exemplary embodiment, a problem in a prior art will be briefly described. Propylene carbonate has slightly high viscosity of 2.5 mPa·s at room temperature, and has a problem that a resistance value of an element increases particularly at low temperature. Acetonitrile has low viscosity at room temperature, but there is a possibility that hydrogen cyanide gas is generated due to combustion or the like in the event of an accident, and its use is limited due to safety problems.

Hereinafter, an exemplary embodiment of electrolytic solution and an electricity storage element according to the present disclosure will be described with reference to an example, but the present disclosure is not limited to the example described below.

Although specific numerical values and materials may be provided as examples in the description below, other numerical values and materials may be applied as long as an effect of the present disclosure can be obtained. In description below, in a case where lower limits and upper limits of numerical values related to specific physical properties, conditions, or the like are illustrated, any of the illustrated lower limits and any of the illustrated upper limits can be optionally combined unless the lower limit is equal to or more than the upper limit. In a case where a plurality of materials are illustrated, one type of the materials may be selected and used alone, or two or more types of the materials may be used in combination.

Examples of the electricity storage element include nonaqueous electrolytic solution capacitor and nonaqueous electrolytic secondary battery. The electricity storage element may be an element that uses both a Faradaic reaction and a non-Faradaic reaction (that is, has properties of both a capacitor and a secondary battery). Examples of the nonaqueous electrolytic solution capacitor include an electric double layer capacitor, a lithium ion capacitor, and the like. Examples of the non-aqueous electrolytic secondary battery include a lithium ion secondary battery, a lithium metal secondary battery, and the like. The capacitor may be referred to as a “capacitor”.

Electrolytic solution according to one exemplary embodiment of the present disclosure is nonaqueous electrolytic solution, and includes non-aqueous solvent and electrolyte salt dissolved in non-aqueous solvent. The non-aqueous solvent may be organic solvent. The non-aqueous solvent contains a first compound. The first compound is at least one selected from the group consisting of 1,3-diethyl-4-methyl-1-cyclobutene and its fluoro-substituted compound. The first compound has low viscosity, and may have viscosity of 0.6 mPa·s or less at room temperature. Further, since the first compound does not contain a cyano group, the first compound does not generate toxic hydrogen cyanide gas even when burned. When the non-aqueous solvent contains the first compound, the electricity storage element can exhibit excellent electrical characteristics even at low temperature. Specifically, there are provided a safe nonaqueous electrolytic solution capacitor, a nonaqueous electrolytic solution secondary battery, and the like which have low internal resistance, are excellent in conductivity, and do not generate toxic gas when burned.

1,3-diethyl-4-methyl-1-cyclobutene has a structure represented by Formula (1).

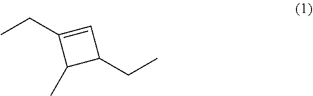

The first compound is at least one selected from the group consisting of 1,3-diethyl-4-methyl-1-cyclobutene and its fluoro-substituted compound, and the fluoro-substituted compound of 1,3-diethyl-4-methyl-1-cyclobutene is a compound in which one or more optional hydrogen atoms of hydrogen atoms of 1,3-diethyl-4-methyl-1-cyclobutene are substituted with a fluorine atom.

Since the first compound is contained in non-aqueous solvent, viscosity becomes lower, so that resistance of the electricity storage element at low temperature can be further reduced.

Content of the first compound in the non-aqueous solvent preferably ranges from 5% by mass to 80% by mass, inclusive. When the content of the first compound is 5% by mass or more, viscosity of the entire mixed non-aqueous solvent is sufficiently reduced, and resistance at low temperature is sufficiently improved. On the other hand, when the content of the first compound is 80% by mass or less, precipitation of electrolyte salt (for example, quaternary ammonium salt, lithium salt, and the like) is suppressed, and characteristics of the electricity storage element are further improved.

Electrolytic solution of the present disclosure may be electrolytic solution in which at least one selected from the group consisting of quaternary ammonium salt and lithium salt is dissolved in non-aqueous solvent. Electrolytic solution of the electric double layer capacitor may contain quaternary ammonium salt. The lithium ion capacitor, the lithium ion secondary battery, the lithium metal secondary battery, and the like may contain lithium salt.

The non-aqueous solvent may contain another compound in addition to the first compound. As the another compound, a second compound that is at least one selected from the group consisting of cyclic carboxylic acid ester, chain carboxylic acid ester, cyclic carbonate ester, chain carbonate ester, and cyclic sulfone compound can be used.

Examples of the cyclic carboxylic acid ester include α-acetolactone, β-propiolactone, γ-butyrolactone, and δ-valerolactone, and γ-butyrolactone is particularly preferable. Examples of the cyclic carboxylic acid ester include methyl acetate, ethyl acetate, methyl propionate, and the like. Examples of the cyclic carbonate ester include vinylene carbonate, propylene carbonate, ethylene carbonate, butylene carbonate, and the like. Examples of the chain carbonate ester include dimethyl carbonate, methyl ethyl carbonate, diethyl carbonate, and the like. Examples of the cyclic sulfone compound include sulfolane, and alkylsulfolane, and 3-methylsulfolane is particularly preferable.

As the quaternary ammonium salt, salt composed of a tetraalkylammonium ion and an anion is desirable.

As the tetraalkylammonium ion, at least one a tetramethylammonium ion, a trimethylethylammonium ion, a triethylmethylammonium ion, a tetraethylammonium ion, a tetrabutylammonium ion, a diethyldimethylammonium ion, and the like can be used.

Examples of the anion constituting the quaternary ammonium salt or the lithium salt include Cl⁻, BF₄⁻, PF⁻, ClCO₄⁻, CF₃SO₃⁻, N(FSO₂)₂⁻, N(CF₃SO₂)₂⁻, N(C₂F₅SO₂)₂⁻, C(CF₃SO₂)₃⁻, and the like.

Specific examples of the quaternary ammonium salt include triethylmethylammonium tetrafluoroborate, and specific examples of the lithium salt include LiPF₆, LiBF₄, and LiN(FSO₂)₂, and the like.

A preferred lower limit of concentration of electrolyte salt in electrolytic solution of the present disclosure is 0.1 mol/L, and a preferred upper limit is 3.0 mol/L. When concentration of electrolyte salt is more than or equal to 0.1 mol/L, sufficient conductivity can be secured. When concentration of electrolyte salt is less than or equal to 3.0 mol/L, increase in viscosity of obtained electrolytic solution can be suppressed, and an electricity storage element having excellent electrical characteristics can be obtained. A lower limit of concentration of the electrolyte salt is more preferably 0.5 mol/L, and an upper limit of the concentration is more preferably 2 mol/L.

A method of manufacturing the electrolytic solution of the present disclosure is as described later. First, the non-aqueous solvent and the electrolyte salt (quaternary ammonium salt, lithium salt, and the like) are dehydrated. After the above, in a low-humidity environment such as a glove box, electrolyte salt containing at least type selected from the group consisting of quaternary ammonium salt and lithium salt is added to the non-aqueous solvent, and the non-aqueous solvent is dissolved.

Furthermore, an electricity storage element using the electrolytic solution prepared here is also included in the present disclosure. For example, the electric double layer capacitor includes a pair of polarizable electrodes, a separator interposed between electrodes, electrolytic solution, and a container that seals these. The lithium ion capacitor includes a polarizable positive electrode, a negative electrode into and from which a lithium ion can be inserted and extracted, electrolytic solution, a separator interposed between electrodes, and a container that houses these. The lithium ion secondary battery includes a positive electrode into and from which a lithium ion can be inserted and extracted, a negative electrode into and from which a lithium ion can be inserted and extracted, electrolytic solution, a separator interposed between electrodes, and a container that houses these.

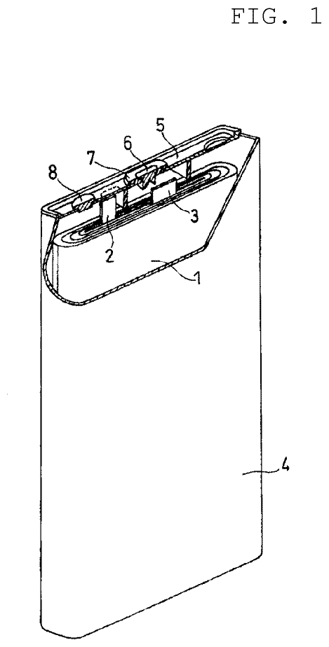

Hereinafter, as an example of the electricity storage element, a structure of a nonaqueous electrolyte secondary battery will be described with reference to FIG. 1. FIG. 1 is a schematic perspective view in which a part of a rectangular nonaqueous electrolyte secondary battery is cut away.

The secondary battery includes bottomed rectangular battery case 4, and electrode group 1 and nonaqueous electrolyte (not illustrated) housed in battery case 4. Electrode group 1 includes an elongated strip-shaped negative electrode, an elongated strip-shaped positive electrode, and a separator interposed between them. Electrode group 1 is formed by winding the negative electrode, the positive electrode, and the separator around a flat plate-shaped winding core, and removing the winding core.

One end of negative electrode lead 3 is attached to a negative current collector of the negative electrode by welding or the like. One end of positive electrode lead 2 is attached to a positive current collector of the positive electrode by welding or the like. Another end of negative electrode lead 3 is electrically connected to negative electrode terminal 6 provided on sealing plate 5 with gasket 7 interposed between them. Another end of positive electrode lead 2 is electrically connected to battery case 4 also serving as a positive electrode terminal. A resin-made frame body that isolates electrode group 1 from sealing plate 5 and isolates negative electrode lead 3 from battery case 4 is disposed above electrode group 1. An opening of battery case 4 is sealed with sealing plate 5.

In the present disclosure, as another exemplary embodiment, at least one compound selected from the group consisting of 1,3-diethyl-4-methyl-1-cyclobutene represented by Formula (1) and its fluoro-substituted compound is included.

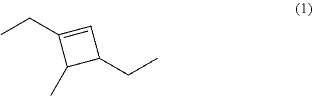

Furthermore, in the present disclosure, as another exemplary embodiment, an additive agent for electrolytic solution containing at least type selected from the group consisting of
1,3-diethyl-4-methyl-1-cyclobutene represented by Formula (1) and its fluoro-substituted compound is included.

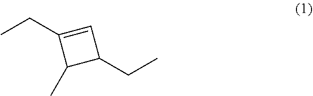

### Example

Although the present disclosure will be specifically described below based on Examples and Comparative Examples, the present disclosure is not limited to Examples below.

### Method of manufacturing 1,3-diethyl-4-methyl-1-cyclobutene

The compound 1,3-diethyl-4-methyl-1-cyclobutene is synthesized by a cyclobutene ring-forming reaction by a [2+2]cycloaddition reaction of alkyne and alkene.

Dichloromethane (1.2 L) was added to [1,1-bis(diphenylphosphino) ferrocene]dichlorocobalt (II) (12.5 g, 0.023 mol) heated and dried under an inert gas atmosphere, and the mixture was stirred at 0° C. for five minutes, and then, trimethylaluminum-toluene solution (2M) (66.6 g, 0.462 mol) was slowly added, and the mixture was stirred at 0° C. for 15 minutes. The inside of a reaction vessel was carefully evacuated, then 1-butyne gas was introduced little by little while maintaining 0° C., and, after that, the mixture was stirred for 30 minutes. After the reaction solution was adjusted to −30° C., cis-2-pentene (32.4 g, 0.462 mol) was slowly added dropwise over 30 minutes, and the mixture was stirred at room temperature for three hours. The reaction solution was diluted with pentane, quenched with methanol, and then subjected to Celite filtration, and solvent was removed from the filtrate under reduced pressure. The obtained residue was purified by silica gel column chromatography (pentane 100%) to obtain 1,3-diethyl-4-methyl-1-cyclobutene (43.0 g, 0.346 mol) in a yield of 75%.

### (Measurement of Viscosity)

Viscosity of 1,3-diethyl-4-methyl-1-cyclobutene synthesized as described above at room temperature (25° C.) was measured with a viscometer RSM-MV1 manufactured by SMILECo. The viscosity was 0.49 mPa·s, and was 0.6 mPa·s or less. This value is lower than 2.5 mPa·s, which is the viscosity of propylene carbonate generally and frequently used as solvent for electrolytic solution. Further, diethyl carbonate and dimethyl carbonate are also often used as low viscosity solvent, and their viscosities are 0.8 mPa·s and 0.6 mPa·s, respectively, and the above viscosity is lower than these.

### Comparative Example 1

Triethylmethylammonium tetrafluoroborate was added to propylene carbonate so as to have concentration of 1.0 mol/L to obtain electrolytic solution for a capacitor.

### Comparative Example 2

Triethylmethylammonium tetrafluoroborate was added to solvent obtained by mixing 90 parts by weight of propylene carbonate and 10 parts by weight of dimethyl carbonate so as to have concentration of 1.0 mol/L to obtain electrolytic solution for a capacitor.

### Example 1

Triethylmethylammonium tetrafluoroborate was added to solvent obtained by mixing 90 parts by weight of propylene carbonate and 10 parts by weight of 1,3-diethyl-4-methyl-1-cyclobutene so as to have concentration of 1.0 mol/L, and electrolytic solution for a capacitor was obtained.

### <Preparation of Laminate Cell>

An aluminum sheet having width of 30 mm and thickness of 20 m was prepared as a current collector, and both surfaces of the current collector were coated with activated carbon having thickness of 80 m to prepare an electrode. Then, the electrode was cut into 20×72 mm, and an electrode lead was welded to an aluminum surface of the current collector. A separator made from cellulose having thickness of 50 m was sandwiched between a pair of electrodes and housed in a container made from an aluminum laminate film, and electrolytic solution was injected in a dry chamber and impregnated into the electrodes. After the above, the container was sealed to produce a laminate cell of a capacitor.

### <Measurement of Internal Resistance>

Voltage of 3.0 V was applied to a produced capacitor, and internal resistance of the capacitor was measured at −30° C.

Table 1 shows a relative value of a measured internal resistance value of each laminate cell at −30° C. to an internal resistance value of Comparative Example 1.

TABLE 1

| | Relative value to |
|---|---|
| | Comparative Example 1 |
| Comparative Example 1 | 1.0 |
| Comparative Example 2 | 0.94 |
| Example 1 | 0.89 |

From a result in Table 1, it is found that an internal resistance value in Example 1 using 1,3-diethyl-4-methyl-1-cyclobutene is lower than that in Comparative Example 1 using only propylene carbonate.

Further, as in the case of Comparative Example 2, it is found that the internal resistance value is reduced as compared with a system in which dimethyl carbonate is added to propylene carbonate.

### <Preparation of Secondary Battery>

### (Negative Electrode)

A negative electrode active material (graphite), sodium carboxymethylcellulose (CMC-Na), and styrene-butadiene rubber (SBR) were mixed at a mass ratio of 97.5:1:1.5, water was added to the mixture, and then the mixture was stirred using a mixer (T.K.HIVIS MIX manufactured by PRIMIX Corporation) to prepare slurry of a negative electrode mixture. Next, the slurry of a negative electrode mixture was applied to a surface of copper foil so that mass of the negative electrode mixture per 1 m² was 190 g, and the coating film was dried and then rolled to prepare a negative electrode in which a negative electrode mixture layer having density of 1.5 g/cm³ was formed on both surfaces of the copper foil.

### (Positive Electrode)

A lithium nickel composite oxide (LiNi_{0.8}Co_{0.18}Al_{0.02}O₂), acetylene black, and polyvinylidene fluoride were mixed at a mass ratio of 95:2.5:2.5, N-methyl-2-pyrrolidone (NMP) was added, and then the mixture was stirred using a mixer (T.K.HIVIS MIX manufactured by PRIMIX Corporation) to prepare slurry of a positive electrode mixture. Next, the slurry of a positive electrode mixture was applied to a surface of aluminum foil, and the coating film was dried and then rolled to prepare a positive electrode in which a positive electrode mixture layer having density of 3.6 g/cm³ was formed on both surfaces of the aluminum foil.

### Comparative Example 3

Ethylene carbonate (EC), dimethyl carbonate (DMC), and ethyl methyl carbonate (EMC) were mixed at a volume ratio of 20:70:10 to prepare a nonaqueous electrolytic solution. LiPF₆ was used as the lithium salt. Concentration of LiPF₆ in the electrolytic solution was 1.2 mol/L.

### Example 4

Ethylene carbonate (EC), dimethyl carbonate (DMC), ethyl methyl carbonate (EMC), and 1,3-diethyl-4-methyl-1-cyclobutene were mixed at a volume ratio of 18:63:9:10 to prepare a nonaqueous electrolytic solution. LiPF₆ was used as the lithium salt. Concentration of LiPF₆ in the electrolytic solution was 1.2 mol/L.

A tab was attached to each electrode, and a positive electrode and a negative electrode were spirally wound with a separator interposed between them in a manner that the tab was positioned at an outermost periphery, so that an electrode group was prepared. The electrode group was inserted into an exterior body made from an aluminum laminate film, vacuum-dried at 105° C. for two hours, and, after that, nonaqueous electrolytic solution was injected and an opening of the exterior body was sealed so as to obtain a secondary battery.

### <Measurement of Discharge Capacity (Battery Capacity)>

In the secondary battery produced as described above, constant current charge was performed with current of 0.3 It (800 mA) under an environment of −5° C. until voltage reached 4.2 V, and, after that, constant voltage charge was performed with constant voltage of 4.2 V until current reached 0.015 It (40 mA). After the above, constant current discharge was performed with current of 0.3 It (800 mA) until voltage reached 2.75 V. Discharge capacity at this time was obtained as battery capacity.

A relative value of battery capacity at −5° C. of each secondary battery measured in the above manner to battery capacity of Comparative Example 3 is shown in Table 2.

TABLE 2

| | Relative value to |
|---|---|
| | Comparative Example 3 |
| Comparative Example 3 | 1.0 |
| Example 4 | 1.1 |

From a result in Table 2, it is found that in Example 4 using 1,3-diethyl-4-methyl-1-cyclobutene, battery capacity is increased as compared with Comparative Example 3 using electrolytic solution not containing such a compound.

It can be seen that by using the electrolytic solution material according to the present disclosure, internal resistance of an element can be reduced, and an operation characteristic at low temperature can be improved.

### <<Appendix>>

The above description of the exemplary embodiment discloses a technique below.

### (Technique 1)

Electrolytic solution comprising:
a non-aqueous solvent; and
an electrolyte salt dissolved in the non-aqueous solvent, wherein:
the non-aqueous solvent contains a first compound, and
the first compound is at least one selected from the group consisting of 1,3-diethyl-4-methyl-1-cyclobutene represented by the following Formula (1) and its fluoro-substituted compound.

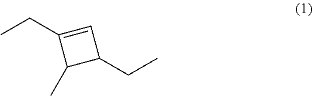

### (Technique 2)

The electrolytic solution according to Technique 1, in which a content of the first compound in the non-aqueous solvent ranges from 5% by mass to 80% by mass, inclusive.

### (Technique 3)

The electrolytic solution according to Technique 1 or 2, in which the non-aqueous solvent further contains a second compound selected from the group consisting of cyclic carboxylic acid ester, chain carboxylic acid ester, cyclic carbonate ester, chain carbonate ester, and cyclic sulfone compound.

### (Technique 4)

The electrolytic solution according to any one of Techniques 1 to 3, in which the electrolyte salt contains at least one selected from the group consisting of quaternary ammonium salt and lithium salt.

### (Technique 5)

The electrolytic solution according to Technique 4, in which the quaternary ammonium salt contains a salt containing a tetraalkylammonium ion and an anion.

### (Technique 6)

The electrolytic solution according to Technique 5, in which the tetraalkylammonium ion is at least one selected from the group consisting of a tetramethylammonium ion, a trimethylethylammonium ion, a triethylmethylammonium ion, a tetraethylammonium ion, a tetrabutylammonium ion, and a diethyldimethylammonium ion.

### (Technique 7)

The electrolytic solution according to Technique 5 or 6, in which the anion is at least one selected from the group consisting of Cl⁻, BF₄⁻, PF₆⁻, ClCO₄⁻, CF₃SO₃⁻, N(FSO₂)₂⁻, N(CF₃SO₂)₂⁻, N(C₂F₅SO₂)₂⁻, and C(CF₃SO₂)₃⁻.

### (Technique 8)

The electrolytic solution according to Technique 4, in which the quaternary ammonium salt is triethylmethylammonium tetrafluoroborate, and the lithium salt is LiPF₆.

### (Technique 9)

The electrolytic solution according to any one of Techniques 1 to 8, in which, in the electrolytic solution, a concentration of the electrolyte salt ranges from 0.1 mol/L to 3.0 mol/L inclusive.

### (Technique 10)

The electrolytic solution according to any one of Techniques 1 to 8, in which, in the electrolytic solution, a concentration of the electrolyte salt ranges from 0.5 mol/L to 2.0 mol/L inclusive.

### (Technique 11)

An electricity storage element including the electrolytic solution according to any one of Techniques 1 to 10.

### (Technique 12)

At least one compound selected from the group consisting of 1,3-diethyl-4-methyl-1-cyclobutene represented by the following Formula (1) and its fluoro-substituted compound.

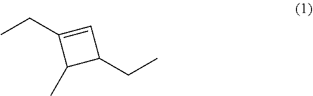

### (Technique 13)

An additive agent for electrolytic solution containing at least one selected from the group consisting of 1,3-diethyl-4-methyl-1-cyclobutene represented by the following Formula (1) and its fluoro-substituted compound.

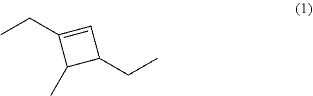

### INDUSTRIAL APPLICABILITY

The electrolytic solution according to the present disclosure is used for an electricity storage element such as a nonaqueous electrolytic solution capacitor, a nonaqueous electrolytic secondary battery, or the like. The electricity storage element according to the present disclosure is useful as a main power supply for a mobile communication device, a mobile electronic device, and the like.

### REFERENCE MARKS IN THE DRAWINGS

1 electrode group
2 positive electrode lead
3 negative electrode lead
4 battery case
5 sealing plate
6 negative electrode terminal
7 gasket

1. Electrolytic solution comprising:
a non-aqueous solvent; and
an electrolyte salt dissolved in the non-aqueous solvent, wherein:
the non-aqueous solvent contains a first compound, and
the first compound is at least one selected from the group consisting of 1,3-diethyl-4-methyl-1-cyclobutene represented by following Formula (1) and a fluoro-substituted compound of 1,3-diethyl-4-methyl-1-cyclobutene.

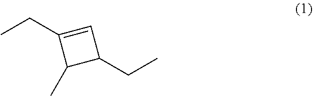

2. The electrolytic solution according to claim 1, wherein a content of the first compound in the non-aqueous solvent ranges from 5% by mass to 80% by mass, inclusive.

3. The electrolytic solution according to claim 1, wherein the non-aqueous solvent further contains a second compound selected from the group consisting of cyclic carboxylic acid ester, chain carboxylic acid ester, cyclic carbonate ester, chain carbonate ester, and cyclic sulfone compound.

4. The electrolytic solution according to claim 1, wherein the electrolyte salt contains at least one selected from the group consisting of quaternary ammonium salt and lithium salt.

5. The electrolytic solution according to claim 4, wherein the quaternary ammonium salt contains a salt containing a tetraalkylammonium ion and an anion.

6. The electrolytic solution according to claim 5, wherein the tetraalkylammonium ion is at least one selected from the group consisting of a tetramethylammonium ion, a trimethylethylammonium ion, a triethylmethylammonium ion, a tetraethylammonium ion, a tetrabutylammonium ion, and a diethyldimethylammonium ion.

7. The electrolytic solution according to claim 5, wherein the anion is at least one selected from the group consisting of Cl⁻, BF₄⁻, PF₆⁻, ClCO₄⁻, CF₃SO₃⁻, N(FSO₂)₂⁻, N(CF₃SO₂)₂⁻, N(C₂F₅SO₂)₂⁻, and C(CF₃SO₂)₃⁻.

8. The electrolytic solution according to claim 4, wherein:
the quaternary ammonium salt is triethylmethylammonium tetrafluoroborate, and
the lithium salt is LiPF₆.

9. The electrolytic solution according to claim 4, wherein in the electrolytic solution, a concentration of the electrolyte salt ranges from 0.1 mol/L to 3.0 mol/L, inclusive.

10. The electrolytic solution according to claim 4, wherein in the electrolytic solution, a concentration of the electrolyte salt ranges from 0.5 mol/L to 2.0 mol/L, inclusive.

11. An electricity storage element comprising the electrolytic solution according to claim 1.

12. At least one compound selected from the group consisting of 1,3-diethyl-4-methyl-1-cyclobutene represented by following Formula (1) and a fluoro-substituted compound of 1,3-diethyl-4-methyl-1-cyclobutene. [chemical structure]
